(19) United States
(12) Patent Application Publication
(10) Pub. No.: US 2024/0270776 A1
(43) Pub. Date: Aug. 15, 2024

(54) PALLADIUM VINYL AND BUTADIENYL COMPOUNDS AS CHEMOTHERAPEUTIC AGENTS

(71) Applicant: UNIVERSITÀ CA' FOSCARI VENEZIA, Venezia (IT)

(72) Inventors: Fabiano VISENTIN, Venezia (IT); Flavio RIZZOLIO, Venezia (IT); Thomas SCATTOLIN, Venezia (IT); Dario ALESSI, Venezia (IT)

(73) Assignee: UNIVERSITÀ CA' FOSCARI VENEZIA, Venezia (IT)

(21) Appl. No.: 18/560,178

(22) PCT Filed: May 2, 2022
(86) PCT No.: PCT/EP2022/061725

§ 371 Date: Nov. 10, 2023

(30) Foreign Application Priority Data
May 11, 2021 (IT) 102021000012107

(51) Int. Cl. C07F 15/00; A61K 31/555; A61K 31/66; A61P 35/00

(52) U.S. Cl. CPC C07F 15/006; A61K 31/555; A61K 31/66; A61P 35/00

(57) ABSTRACT

The present invention relates to chemotherapeutic compounds and, in particular, metal complexes used for the purpose. Furthermore, it also relates to an innovative process for the preparation of said compounds and their therapeutic use.

### TECHNICAL FIELD

The present invention relates to chemotherapeutic compounds and, in particular, metal complexes used for the purpose. Furthermore, it also relates to an innovative process for the preparation of said compounds and their therapeutic use.

### BACKGROUND OF THE INVENTION

About half of current chemotherapy treatments are based on Platinum compounds, a metal at the base of anticancer substances such as cisplatin (cis-diaminodichloroplatin (II)) and its second and third generation derivatives (carboplatin and oxaliplatin), in use since the end of the 70s despite having, in some cases, some important critical issues, linked on the one hand to their neuro-, nephro- and ototoxicity, on the other to their ineffectiveness towards certain types of tumors.

For this reason, many researchers have recently oriented their studies towards the synthesis and study of the anticancer properties of compounds containing metals other than Platinum, among which especially Ruthenium and Gold have played an important role.

An advantage of the Palladium derivatives with respect to the Platinum homologs is represented by their greater solubility in water, while a contraindication is their lower inertia (in particular the exchange rates of the coordinated ligands are about 10⁵ times higher) which may have of the negative repercussions on their stability in the biological environment.

It was also possible to ascertain that the antiproliferative activity towards tumor cells was carried out with diversified mechanisms of action. Palladium derivatives sometimes interact with DNA, similarly to cisplatin, distorting its configuration or damaging it, forming cross-linked intermediates; alternatively, they can influence the activity of some enzymes that supervise the processes of apoptosis or interfere with DNA repair mechanisms. A further possibility is represented by the induction of dysfunctions at the mitochondrial level.

Despite the great variety of mechanisms of action, what appears evident however is the importance of coordinated ligands on the metal center which play a fundamental role in determining the geometry of the complexes, in stabilizing a specific oxidation state, modulate the lipophilic or hydrophilic properties of the compounds, all aspects that can heavily influence their pharmacological efficacy and/or the possibility of limiting their unwanted side effects.

A category of ligands recently much studied in this regard are the N-heterocyclic carbenes (NHCs), which are, for example, object of patent application PCT/EP2020/084360.

Palladium vinyl and butadienyl compounds have been the object of numerous studies in the past, as they are involved in catalytic processes of oligomerization and polymerization of alkynes. Examples of complexes of this type are described in the articles by Vicenzo De Felice, Maria E. Cucciolito, Augusto De Renzi, Francesco Ruffo, Diego Tesauro, Journal of Organometallic Chemistry 1995, 493, 1-11 and by Luciano Canovese, Fabiano Visentin, Gavino Chessa, Paolo Uguagliati, Claudio Santo, Alessandro Dolmella, Organometallics 2005, 24, 3297-3308 which discloses the compound of formula:

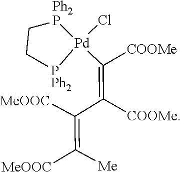

The publication in the name of Thomas Scattolin, Isabella Caligiuri, Nayla Mouwad, Maguie El Boustani, Nicola Demitri, Flavio Rizzolio, Fabiano Visentin, in European Journal of Medicinal Chemistry, 179 (2019), 325-334, discloses the compound of formula 1a:

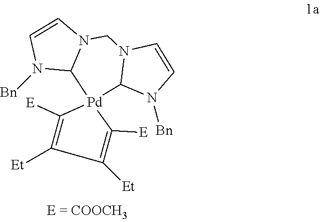

The synthesis of this category of metal derivatives generally involves the insertion of functionalized alkynes on the Pd—C bond as shown in scheme 1. The spectator ligands used are generally chelators (L-L′) with equal or different donor atoms, typically phosphorus, nitrogen and sulfur. The R group involved in migratory insertion may instead be an alkyl, an aryl or a vinyl, while to activate the process it requires Z substituents on the alkynes with a decidedly electron-atractor character (e.g. —COOR, —CF₃).

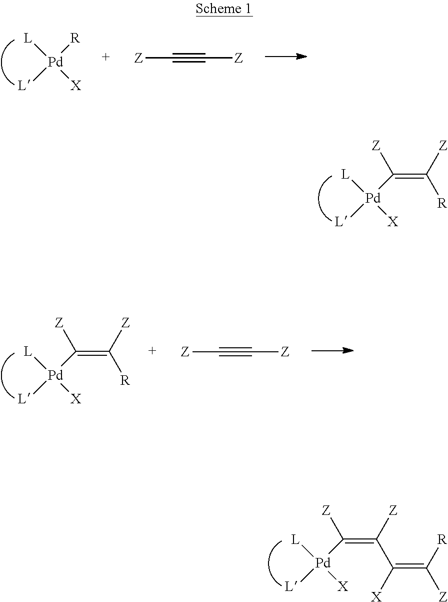

### SUMMARY OF THE INVENTION

The problem addressed by the present invention is therefore that of providing a new compound useful as an antitumor agent.

This problem is solved by the compound of formula (A), or of formula (B) or of formula (C) and by the relative use as outlined by the attached claims, the definitions of which are an integral part of the present description.

In particular, the invention relates to a compound that can be used as an antitumor agent and/or in a method for the treatment of tumors.

A further aspect is an innovative process for the preparation of said compound which sees the reaction of a compound of formula D with suitable L and/or L′ ligands or with L-L′ bidentates.

Further characteristics and advantages of the use of the compound of the invention, of its medical use, of the process for its preparation will result from the description of the examples of the invention, provided as an indication of the invention.

### BRIEF DESCRIPTION OF THE DRAWINGS

FIGS. 1-A, 1-B and 1-C respectively show ¹H-NMR, ¹³C{¹H}-NMR and ³¹P{¹H}-NMR spectra of compound I.

FIGS. 2-A and 2-B respectively show ¹H-NMR and ¹³C{¹H}-NMR spectra of compound V.

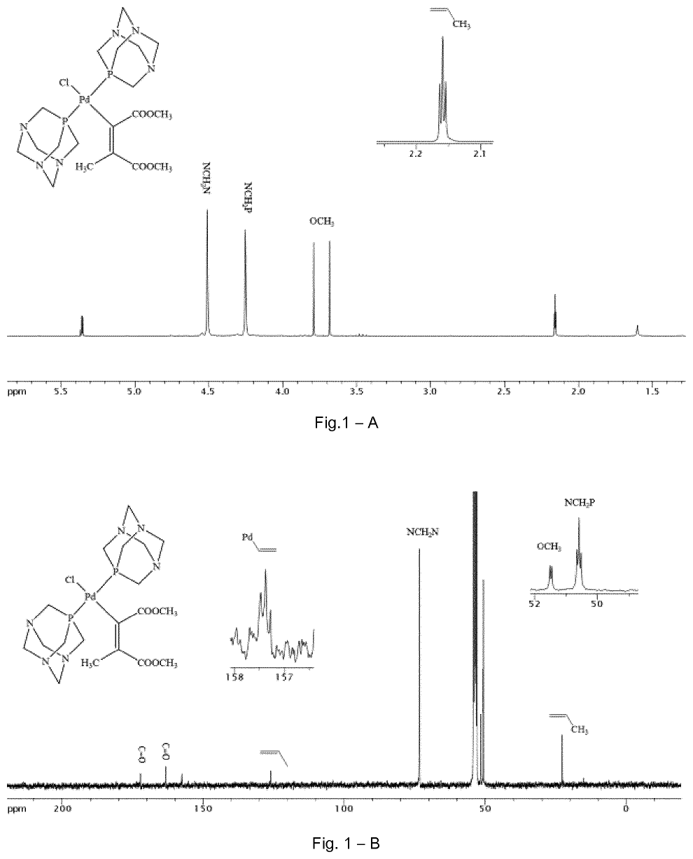

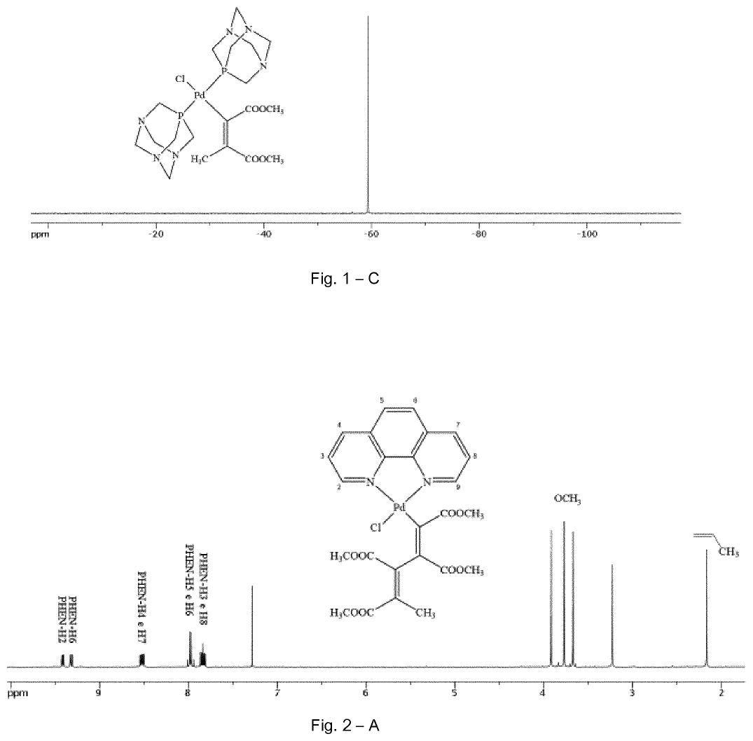

In the proton spectrum in FIG. 1-A, it is possible seen, for what concerns the vinyl fragment, in addition to those of the two OCH₃ groups, a signal of the terminal CH₃ which resonates as a triplet for the long-range coupling with the two phosphors.

In the carbon spectrum (FIG. 1-B), on the other hand, the two peaks of the carbonyl carbons and those of the two vinyl carbons can be found, of which the one directly linked to Palladium resonates at lower fields (≈157 ppm) as a triplet for coupling with the two phosphors.

The single singlet appearing in the ³¹P{¹H}-NMR spectrum (FIG. 1-C), located at chemical shifts significantly higher than those of free PTA (Δδ≈40 ppm), is an unequivocal proof of trans geometry of the compound, which is punctually confirmed by the presence in the ¹H-NMR and ¹³C{¹H}-NMR spectra (respectively FIGS. 1-A and 1-B), of a single signal for each of the protons and carbons of the NCH₂P and NCH₂N groups.

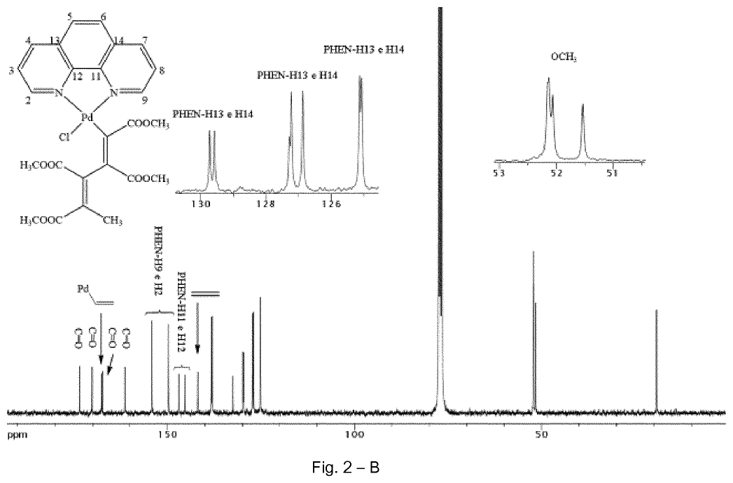

In the ¹H and ¹³C{¹H}NMR spectra (FIGS. 2-A and 2-B) all the signals expected for the bidentate spectator ligand can be found, in a number that agrees with the presence of two different ligands (chloride and the butadienyl residue) in the two remaining coordination sites. A significant increase in chemical shift values should also be noted, especially of the atoms closest to the metal center.

As regards the butadienyl residue, the presence of the signal of the terminal methyl group (with significantly lower chemical shift than in the corresponding vinyls) and the four different OCH₃ groups should be highlighted in the proton spectrum (FIG. 2-A). In the carbon spectrum, there will be found, among others, the four peaks assignable to carbonyl carbons, of which the one directly linked to palladium resonates at lower fields.

### DETAILED DESCRIPTION OF THE INVENTION

An object of the present invention is a compound of formula (A) or formula (B) or formula (C):

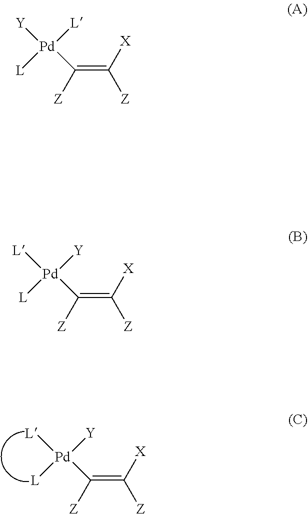

wherein:
X is a halogen,
COOR or R, where R is an alkyl or aryl group,
X is a halogen, COOR or R, where R is an alkyl or aryl group, or X is C(Z)═C(Z)(X′) group, where Z is defined as above, X′ is in geometry trans with respect to Z and is a halogen, COOR or R, where R is an alkyl or aryl group,
L and L′ are equal or different monodentate ligands,
L-L′ is a bidentate ligand;
for use as a medicament.

It has in fact been surprisingly found that the compound of formula (A) or formula (B) or formula (C) can in fact be effectively used in medicine, as a medicament. Said compound has in fact been found to be particularly efficient as an antitumor and/or in an antitumor treatment method.

In other words, the compound of formula (A) or formula (B) or formula (C) is efficient against the tumor, therefore it can be effectively employed as an antitumor agent and/or in a tumor treatment method.

In particular, the compound of formula (A) or formula (B) or formula (C) is efficient against cancer of the ovaries, cervix, colon or lungs; therefore it can be effectively employed as an antitumor agent and/or in a method of treating cancer of the ovaries, cervix, colon or lungs.

Therefore, according to an embodiment, the compound of formula (A) or formula (B) or formula (C) is preferred for use as an antitumor and/or in an antitumor treatment method.

According to one embodiment, the compound of formula (A) or formula (B) or formula (C) is preferred for use in a method of treating tumors of the ovaries, cervix, colon or of the lungs.

In the compound of formula (A) or formula (B) or formula (C), Palladium is in the +2 oxidation state, therefore said compounds can be described as compounds of Palladium (II).

In the compound of formula (A) or formula (B) or formula (C), L and L′ are selected from phosphines, phosphites, isonitriles, amines, thiols, alcohols, carbenes, alkyls, aryl, carbonyl.

In the compound of formula (A) or formula (B) or formula (C), L-L′ is selected from diphosphines, diamines, glycols, biscarbenes, bisisonitriles.

In the compound of formula (A) or formula (B) or formula (C), the R group, whether it belongs to X, X′ or to the COOR group, is an alkyl or aryl group, preferably R is a linear or branched C1-C12 alkyl or aryl group; more preferably R is a linear or branched C1-C6 alkyl group, again more preferably R is a linear or branched C1-C4 alkyl group; finally, according to the most preferred embodiment, R is a methyl group (abbreviated Me).

When R is a linear or branched C1-C4 alkyl, means that R is methyl, ethyl, n-propyl, isopropyl, n-butyl, sec-butyl, isobutyl or tert-butyl.

According to a preferred embodiment, in the compound of formula (A) or formula (B) or formula (C):
Z is COOR, where R is an alkyl group,
X is R, where R is an alkyl group, or X is a group C(Z)═C(Z)(X′), where Z is COOR,
where R is an alkyl group and X′ in trans geometry with respect to Z is an alkyl group.

According to a more preferred embodiment, in the compound of formula (A) or formula (B) or formula (C):
Z is COOMe,
X is Me or X is a group C(Z)═C(Z)(X′), where Z is COOMe and X′ in trans geometry with respect to Z is Me.

In the compound of formula (A) or formula (B) or formula (C), Y is a halogen, therefore it is chosen from among Fluorine, Chlorine, Bromine, Iodine; preferably Y is Chlorine.

According to a preferred embodiment, in the compound of formula (A) or formula (B) or formula (C):
L and/or L′ are chosen between 1,3,5-triaza-7-phosphoadamantane (abbreviated PTA) and triphenylphosphine,
or,
L-L′ is 1,10-phenanthroline, 1,3-bis(diphenylphosphino)propane, 1,2-bis (diphenylphosphino)ethane, or 1,1′-dibenzyl-3,3′-methylenediimidazole-2,2′-diylidene.

PTA is a particular phosphine ligand which is known to be soluble in water and to have the ability to transfer this property to its compounds. This feature, together with the resistance of the PTA to oxidation, helps to make the complex in question more compatible with the biological environment in which it will operate.

According to a preferred embodiment, the compound of formula (A) or formula (B) or formula (C) is selected from the group of the following compounds:

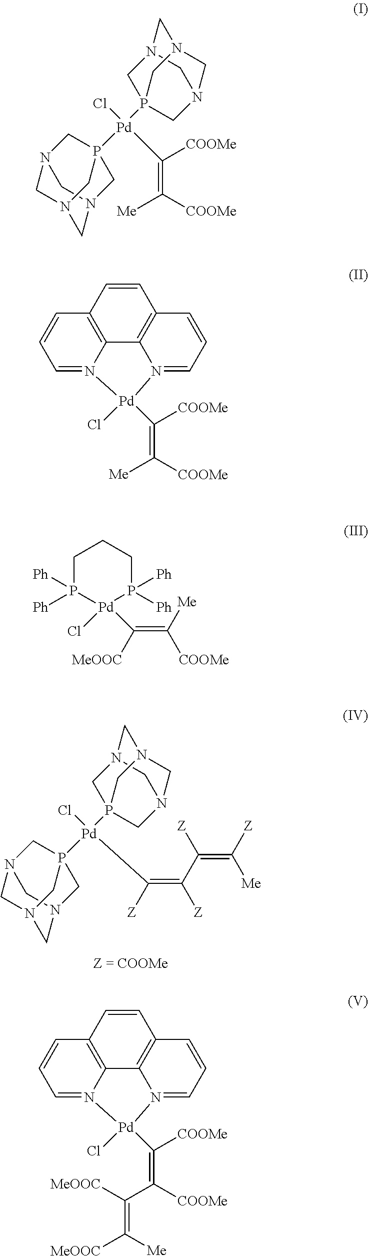

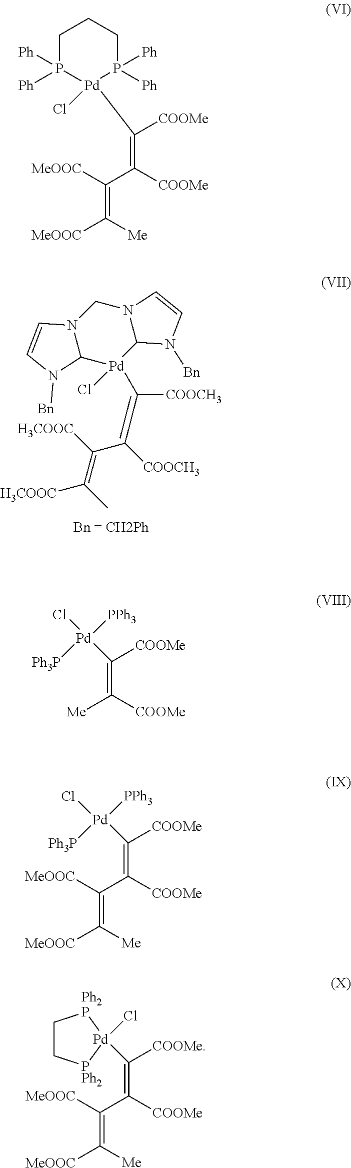

Another object is a pharmaceutical composition comprising a compound of formula (A) or formula (B) or formula (C), including the preferred embodiments described above, and at least one pharmaceutically acceptable excipient.

Said pharmaceutical composition can be in solid form, for example, in tablet form or in liquid form, for example in the form of solution for injection or syrup.

The pharmaceutical composition comprising a compound of formula (A) or formula (B) or formula (C), including the preferred embodiments above described, and at least one pharmaceutically acceptable excipient is for the treatment of the tumor.

Another object is a process for the preparation of a compound of formula (A) or formula (B) or formula (C), including the preferred embodiments above described, comprising the reaction of a compound of formula (D):

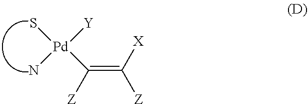

wherein:
Y is a halogen,
Z is COOR or R, where R is an alkyl or aryl group,
X is a halogen, COOR or R, where R is an alkyl or aryl group, or X is a C(Z)═C(Z)(X′) group, where Z is defined as above, X is in geometry trans with respect to Z and is a halogen, COOR or R, where R is an alkyl or aryl group,
S—N is a ligand such as 2-methyl-6-((phenylthio)methyl)pyridine;
with at least one L or L′ monodentate ligand or with an L-L′ bidentate ligand.

In said process, the reaction with two monodentate ligands (L and L′) can lead to the formation of complexes with a cis-(compound of formula B) or trans-(compound of formula A) configuration. The latter configuration is usually predominant.

According to a preferred embodiment of the process, in the compound of formula (D):
Z is COOR, where R is an alkyl group,
X is R, where R is an alkyl group, or X is a group C(Z)═C(Z)(X′), where Z is COOR,
where R is an alkyl group and X′ in trans geometry with respect to Z is an alkyl group.

According to a more preferred embodiment of the process, in the compound of formula (D):
Z is COOMe,
X is Me or X is a group C(Z)═C(Z)(X′), where Z is COOMe and X′ in trans geometry with respect to Z is Me.

According to a preferred embodiment of the process, in the compound of formula (D) Y is Chlorine.

According to a preferred embodiment of the process, the compound (D) is

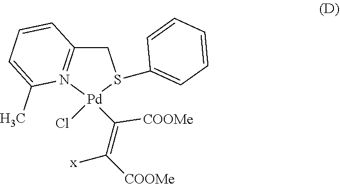

where X is Me or X is a group C(Z)═C(Z)(X′), where Z is COOMe and X′ in trans geometry with respect to Z is Me.

According to a preferred embodiment of the process:
L and/or L′ are chosen from 1,3,5-triaza-7-phosphoadamantane and triphenylphosphine, or,
L-L′ is 1,10-phenanthroline, 1,3-bis (diphenylphosphino) propane, 1,2-bis (diphenylphosphino) ethane, or 1,1′-dibenzyl-3,3′-methylenediimidazole-2,2′-diylidene.

The reaction of the process for the preparation of a compound of formula (A) or of formula (B) or of formula (C) is generally very fast and takes place under mild and very reproducible conditions.

The molar yield of the process is between 78% and 98%.

The reaction of the process for the preparation of a compound of formula (A) or of formula (B) or of formula (C) can be carried out in aqueous solution or in one or more organic solvents. According to a preferred embodiment, the reaction is carried out in dichloromethane.

The reaction of the process for the preparation of a compound of formula (A) or of formula (B) or of formula (C) is preferably carried out at a temperature ranging from 0° C. to 35° C. for a time included from 5 minutes to two hours.

The compound of formula (D) is prepared through synthetic routes such as oxidative addition on Pd (0) complexes or addition of small molecules XY or RX (e.g. halogens and interhalogens, or alkyl halides) to palladacyclopentadienyl complexes or palladium-olefin (see diagram 2), commercially available.

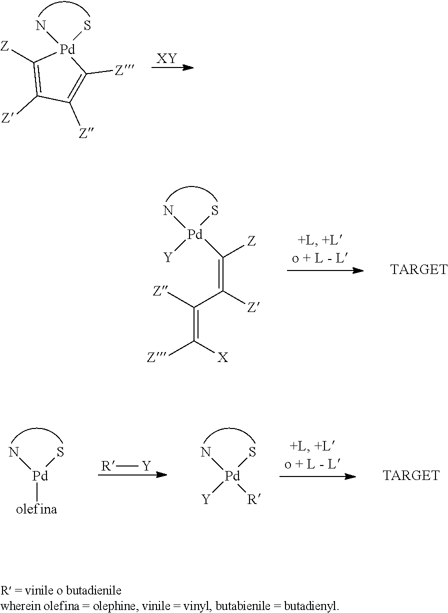

Another aspect is a compound selected from the group of the following compounds:

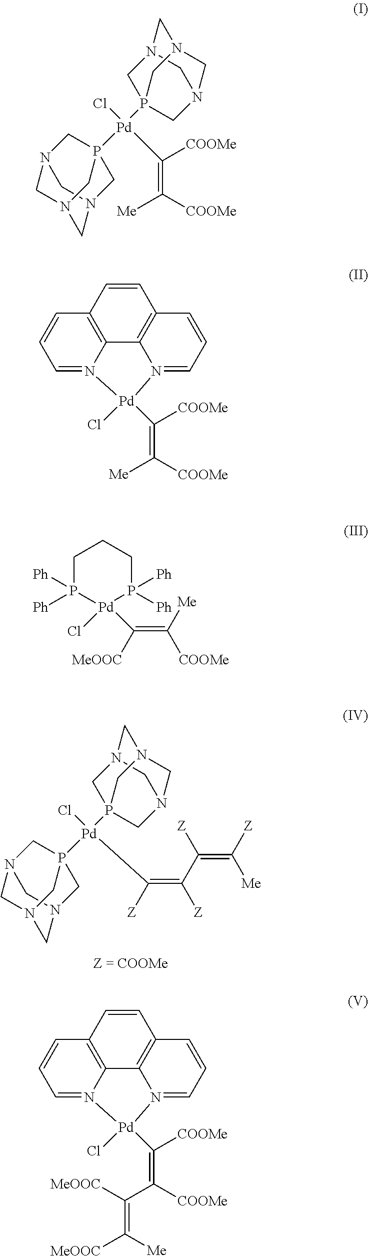

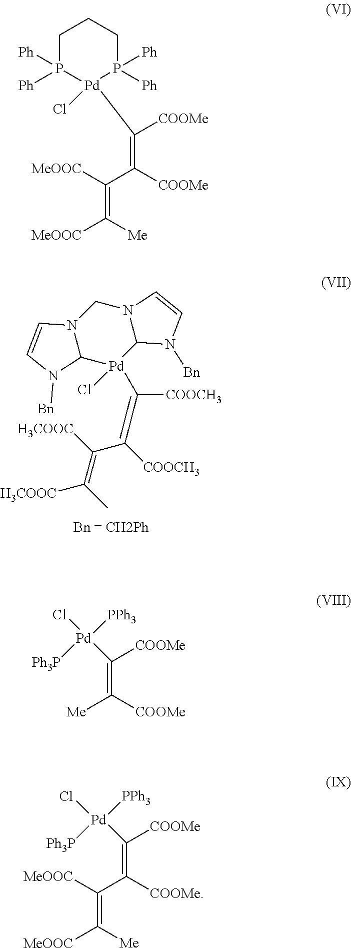

Another aspect is a tumor treatment method comprising administering a compound of formula (A) or formula (B) or formula (C) as described above.

According to a preferred embodiment, the above tumor treatment method comprises the treatment of tumors of the ovaries, cervix, colon or lungs.

According to a preferred embodiment, the above tumor treatment method comprises the administration of a compound chosen from the group of composites of formula I to X.

The following table I constitutes evidence of the effect provided by the compounds of the present invention as an antitumor agent and/or for the tumor treatment method:

TABLE I

| Compound | IC₅₀ (μM) | | | | |
|---|---|---|---|---|---|
| name | A2780 | A2780 cis | MDA-MB231 | DLD1 | MRC-5 |
| CISPLATIN | 0.4 ± 0.1 | 27 ± 5 | 37 ± 9 | 0.63 ± 0.08 | 8 ± 1 |
| Compound I | 0.17 ± 0.04 | 2.2 ± 0.3 | 0.3 ± 0.1 | 0.17 ± 0.03 | >100 |
| Compound II | 3.6 ± 0.2 | 4.2 ± 0.7 | 6.2 ± 0.2 | 2.8 ± 0.8 | 4.5 ± 0.1 |
| Compound III | 1.0 ± 0.3 | 2.0 ± 0.5 | 1.6 ± 0.1 | 4.0 ± 0.6 | 2.9 ± 0.2 |
| Compound IV | 0.08 ± 0.04 | 0.6 ± 0.4 | 0.10 ± 0.01 | 0.06 ± 0.02 | >100 |
| Compound V | 2.7 ± 0.5 | 2.3 ± 0.6 | 3.7 ± 0.5 | 2.4 ± 0.3 | 3.3 ± 0.2 |
| Compound VI | 3.4 ± 0.1 | 5.5 ± 0.8 | 0.5 ± 0.4 | 3.7 ± 0.5 | >100 |
| Compound VII | 1.0 ± 0.2 | 16 ± 3 | 2.0 ± 0.6 | >100 | >100 |
| Compound VIII | 0.117 ± 0.003 | 0.08 ± 0.04 | 0.4 ± 0.3 | 0.7 ± 0.2 | 36 ± 6 |
| Compound IX | 0.13 ± 0.06 | 0.4 ± 0.1 | >100 | 2.7 ± 0.7 | >100 |
| Compound X | 2.8 ± 0.4 | 5 ± 2 | 1.0 ± 0.5 | 4.1 ± 0.8 | >100 |

The values in table (1) were acquired according to the methods and conditions described in Example 11.

The values reported in table (1) show how the compounds of the invention are very active towards all the tumor lines examined: A2780 and A2780cis, (ovarian cancer), MDA-MB231 (triple-negative breast cancer) and DLD1 (colon cancer). Furthermore, all compounds, with the exception of II, Ill and V, were found to be poorly active or inactive (IC50>100 μM) against normal cells (pulmonary fibroblasts MRC-5), thus not presenting the side effects of toxicity found with cisplatin and thus making clear the advantage and effect provided by the compounds of the invention.

### EXPERIMENTAL PART

### Preparation of the Precursor of Formula (D)

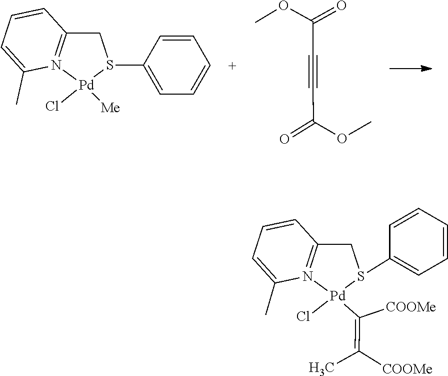

In a 100 mL tailored flask, 20 mL of anhydrous dichloromethane, 0.3002 g (0.8070 mmol) of [(Me-PyCH₂SPh)Pd(Me)Cl] and 109 μL (0.126 g, 0.887 mmol) are introduced in a controlled atmosphere (Ar) of dimethyl-2-butinoate (DMAD). The reaction mixture is left to react under stirring and at room temperature for about 4 hours, during which it is possible to observe the progressive change in its color from an initial pale yellow to light orange. After filtration on millipore to eliminate some solid residue, the solution obtained is reduced to a small volume on the rotary evaporator and the final product is then precipitated by it by adding n-hexane. The orange-pink solid is separated by gooch filtration and dried in a vacuum pump.

0.3978 g (0.7735 mmol) were obtained which correspond to a yield of 96%.

¹H-NMR (300 MHz, CDCl3, T=298 K, ppm) δ: 2.49 (s, 3H, ═CCH₃), 3.15 (s, 3H, py-CH₃), 3.66 (s, 3H, OCH₃), 3.81 (bs, 3H, OCH₃), 4.34 (d, 1H, J=16 Hz, CH₂S); 4.99 (d, 1H, J=16 Hz, CH₂S); 7.07 (d, 1H, J=7.5 Hz, H⁵), 7.15 (d, 1H, J=7.5 Hz, H³), 7.28-7.38 (m, 3H, SPh), 7.56 (t, 1H, J=7.5 Hz, H⁴), 7.67 (bd, 2H, SPh).

### Example 1: Preparation of the Compound of Formula (I)

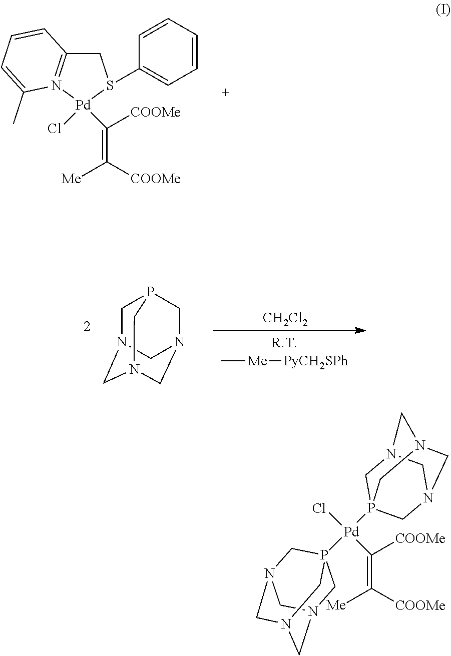

In a 100 mL tailed flask, maintained in an argon atmosphere, 0.0820 g (0.1594 mmol) of the precursor (of formula D) [(Me-PyCH2SPh) Pd (ZC═CZMe) Cl; wherein Z═COOMe] and 0.0526 g (0.3348 mmol) of 1,3,5-triaza-7-phosphoadamantane (PTA). The orange reaction mixture is left to react for one hour. The volume of the solution obtained is then reduced by means of a rotary evaporator. The addition of diethyl ether induces precipitation of the product and the resulting white solid is then separated by filtration on gooch and dried in a vacuum pump. 0.0938 g (0.1529 mmol) equal to 96% yield were obtained.

The process is selective and leads to the exclusive formation of the thermodynamically more stable isomer, having the two phosphine ligands in mutual trans position.

The compound was characterized by NMR and IR spectroscopy.

The ¹H-NMR, ¹³C {1H}-NMR and ³¹P {1H}-NMR spectra of the compound are reported respectively in FIGS. 1-A, 1-B and 1-C. The listing and the attribution of the peaks in the three spectra.

¹H-NMR (300 MHz, CD₂Cl₂, T=298 K, ppm) δ: 2.49 (t, J_{HP}=1.4 Hz, 3H, ═CCH₃), 3.68 (s, 3H, OCH₃), 3.79 (s, 3H, OCH₃), 4.25 (s, 12H, NCH₂P), 4.51 (s, 12H, NCH₂N).

¹³C{¹H}-NMR (T=298K, CD₂Cl₂, ppm) δ: 22.7 (CH₃, ═CCH₃), 50.3 (pseudo-t, CH₂, NCH₂P), 51.4 (CH₃, OCH₃), 51.5 (CH₃, OCH₃), 73.1 (CH₂, NCH₂N), 125.8 (C, ═CCH₃), 157.4 (t, C, J_{CP}=10.2 Hz, PdC═), 163.2 (C, CO), 172.2 (C, CO).

³¹P{¹H}-NMR (T=298K, CD₂Cl₂, ppm) δ: −59.3.

Characteristic IR peaks: ν_{(CO)} 1709 cm⁻¹, in KBr tablet.

### Example 2: Preparation of the Compound of Formula (II)

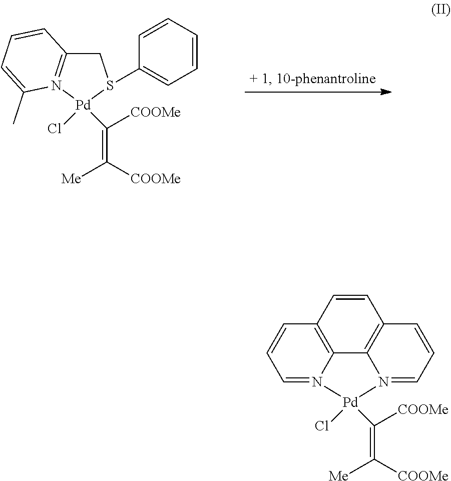

100 mL tailed flask, in an inert atmosphere (Ar), 0.0820 g (0.1594 mmol) of [(Me-PyCH2SPh) Pd (ZC═CZMe) Cl are reacted in 15 mL of anhydrous dichloromethane; wherein Z═COOMe] with 0.0316 g (0.1754 mmol) of 1,10-phenanthroline. The system is left to react under stirring at room temperature for about 30 minutes. The resulting orange solution is filtered on millipore and the volume is reduced by rotary evaporator. The precipitation of the pink product is induced by the addition of diethyl ether. After filtration on gooch, the final solid is dried under vacuum. 0.0753 g (0.1571 mmol) of product corresponding to 98% yield were obtained.

The compound was characterized by NMR and IR spectroscopy.

Below is the listing and the attribution of the peaks in the 1H-NMR and 13C {1H}-NMR spectra.

¹H-NMR (300 MHz, CDCl₃, T=298 K, ppm) δ: 2.56 (s, 3H, ═CCH₃), 3.76 (s, 3H, OCH₃), 3.86 (s, 3H, OCH₃), 7.85-7.93 (m, 2H, phen-H³, phen-H⁸), 8.00-8.02 (AB system, J=8.9 Hz, 2H, phen-H⁵, phen-H⁶), 8.53-8.58 (m, 2H, phen-H⁴, phen-H⁷), 9.31 (dd, J=5.2, 1.4 Hz, phen-H⁹), 9.48 (dd, J=5.0, 1.5 Hz, phen-H²).

¹³C{¹H}-NMR (T=298K, CDCl₃, ppm) δ: 21.5 (CH₃, ═CCH₃), 51.7 (CH₃, OCH₃), 51.9 (CH₃, OCH₃), 125.3, 125.7 (CH, phen-CH³, phen-CH⁸), 127.0, 127.2 (CH, phen-CH⁵, phen-CH⁶), 127.3 (C, ═CCH₃), 129.6, 130.1 (C, phen-C¹³, phen-C¹⁴), 138.1, 138.2 (CH, phen-CH⁴, phen-CH⁷), 145.3, 147.1 (C, phen-C¹¹, phen-CH¹²), 149.6 (CH, phen-CH⁹), 153.3 (CH, phen-CH²), 159.2 (C, PdC═), 163.6 (C, CO), 173.7 (C, CO).

Characteristic IR peack: ν_{(CO)} 1693 cm⁻¹, tablet in KBr.

### Example 3: Preparation of the Compound of Formula (III)

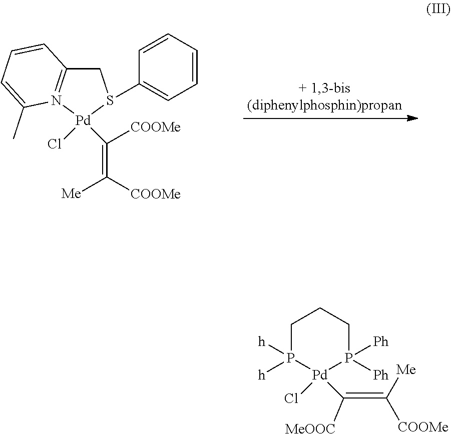

In a 100 mL tailed flask, in a controlled atmosphere (Ar), 0.0820 g (0.1594 mmol) of [(Me-PyCH₂SPh) Pd (ZC═CZMe) Cl are reacted in 12 mL of anhydrous dichloromethane; wherein Z═COOMe] and 0.0745 g (0.1754 mmol) of 1,3-bis (diphenylphosphino) propane. Within a few minutes the solution changes from a yellow to an intense orange color. The system is left to react for about 30 minutes, after which the solution is filtered through millipore and reduced to a small volume. The product is precipitated by adding diethyl ether. After separation on gooch, the pink colored solid is dried in a vacuum line. 0.0888 g (0.1248 mmol) of product were obtained corresponding to 78% yield.

The compound was characterized by NMR and IR spectroscopy.

Below is the listing and the attribution of the peaks in the ¹H-NMR, ¹³C {1H}-NMR and ³¹P {1H}-NMR spectra.

¹H-NMR (300 MHz, CDCl₃, T=298 K, ppm) δ: 1.85 (m, 3H, ═CCH₃), 1.93-2.66 (m, 6H, CH₂), 3.42 (s, 3H, OCH₃), 3.52 (s, 3H, OCH₃), 7.19-7.93 (m, 30H, Ph).

¹³C{¹H}-NMR (T=298K, CDCl₃, ppm) selected peaks, δ:18.8 (CH₃, ═CCH₃), 22.1 (CH₂, CH₂ central), 26.1 (dd, CH₂, J_{CP}=22.6, 4.9 Hz, PCH₂), 27.9 (dd, CH₂, J_{CP}=30.1, 9.0 Hz, PCH₂), 51.0 (CH₃, OCH₃), 51.0 (CH₃, OCH₃), 127.5 (C, ═CCH₃), 170.6 (d,C, J_{CP} 123.9 Hz PdC═), 163.2 (d,C, J_{CP}=15.7, CO), 172.7 (C, CO).

³¹P{¹H}-NMR (T=298K, CDCl₃, ppm) δ: −5.6 (d, J_{CP}=49.1 Hz), 15.3 (d, J_{CP}=49.1 Hz)

Characteristic IR peaks: ν_{(CO)} 1702 cm⁻¹, tablet in KBr

### Example 4: Preparation of the Compound of Formula (IV)

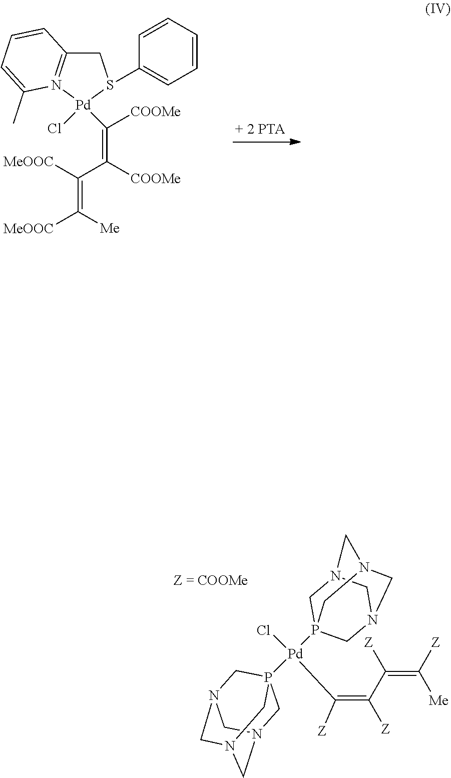

In a 100 mL tailed flask, maintained in a controlled atmosphere (Ar), 0.0820 g (0.1249 mmol) of [(Me-PyCH2SPh) Pd (ZC═CZ—CZ═CZMe) are dissolved in 12 mL of anhydrous dichloromethane Cl; wherein Z═COOMe] and 0.0412 g (0.2623 mmol) of PTA. The resulting orange solution is left to react for half an hour. After this time it is reduced to a small volume and the product is precipitated by adding diethyl ether. The pale orange solid obtained is separated by filtration on gooch and dried in a vacuum pump. 0.0860 g (0.1138 mmol) equal to 91% yield were obtained.

The compound was characterized by NMR and IR spectroscopy.

Below is the listing and the attribution of the peaks in the ¹H-NMR, ¹³C {1H}-NMR and ³¹P {1H}-NMR spectra.

¹H-NMR (300 MHz, CDCl₃, T=298 K, ppm) δ: 1.95 (s, 3H, ═CCH₃), 3.74 (s, 3H, OCH₃), 3.82 (s, 3H, OCH₃), 3.83 (s, 3H, OCH₃), 3.89 (s, 3H, OCH₃), 4.29 (m, 12H, NCH₂P), 4.51 (m, 12H, NCH₂N).

¹³C{¹H}-NMR (T=298K, CDCl₃, ppm) δ: 17.6 (CH₃, ═CCH₃), 50.3 (pseudo-t, CH₂, NCH₂P), 50.0 (CH₃, OCH₃), 52.3 (CH₃, OCH₃), 52.6 (CH₃, OCH₃), 52.9 (CH₃, OCH₃), 73.2 (CH₂, NCH₂N), 127.8 (C, ═CCH₃), 135.4 (C, C═C), 136.4 (C, C═C), 162.4 (C, CO), 168.1 (C, CO), 168.9 (C, CO), 170.3 (t, C, J_{CP}=9.8 Hz, PdC═), 172.4 (C, CO).

³¹P{¹H}-NMR (T=298K, CDCl₃, ppm) δ: −55.8.

Characteristic IR peaks: ν_{(CO)} 1718 cm⁻¹, tablet in KBr

### Example 5: Preparation of the Compound of Formula (V)

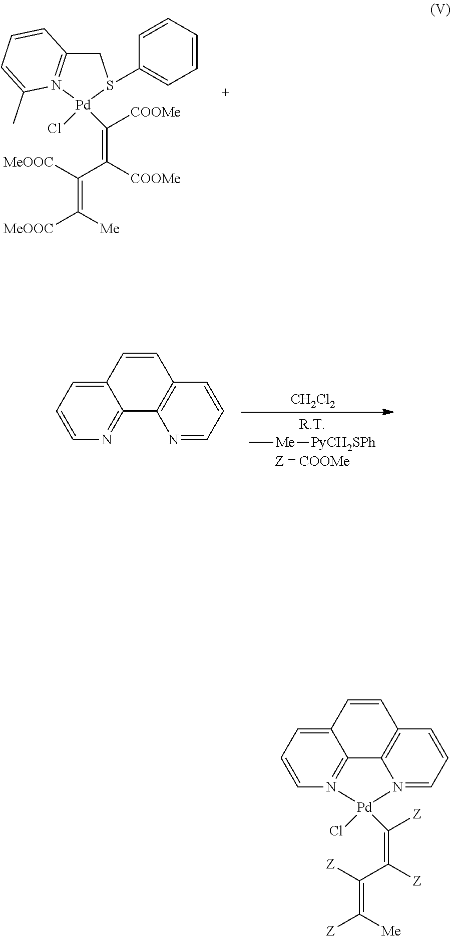

In a 100 mL tailed flask, in an inert atmosphere (Ar), 12 mL of anhydrous dichloromethane, 0.0820 g (0.1249 mmol) of [(Me-PyCH2SPh) Pd (ZC═CZ—CZ═CZMe) Cl are inserted] and 0.0584 g (0.1374 mmol) of 1.10-phenanthroline. The color of the solution in the 30 minutes in which it is left to react, changes from light yellow to a more intense yellow. Finally, the volume of the rotary evaporator is reduced and product precipitation is induced by adding n-hexane. By filtering on gooch, a pink solid is obtained. 0.0692 g (0.1114 mmol) of product were obtained, equal to a yield of 89%.

The compound was characterized by NMR and IR spectroscopy.

The ¹H-NMR and ¹³C {1H}-NMR spectra of the compound are shown respectively in FIGS. 2-A and 2-B. Below is the listing and the attribution of the peaks in the two spectra.

¹H-NMR (300 MHz, CDCl₃, T=298 K, ppm) δ: 2.17 (s, 3H, ═CCH₃), 3.23 (s, 3H, OCH₃), 3.67 (s, 3H, OCH₃), 3.77 (s, 3H, OCH₃), 3.92 (s, 3H, OCH₃), 7.81-7.87 (m, 2H, phen-H³, phen-H⁸), 7.96-7.99 (AB system, J=8.9 Hz, 2H, phen-H⁵, phen-H⁶), 8.50-8.55 (m, 2H, phen-H⁴, phen-H⁷), 9.32 (dd, J=4.6, 1.4 Hz, phen-H⁹), 9.41 (dd, J=4.6, 1.4 Hz, phen-H²).

¹³C{1H}-NMR (T=298K, CDCl₃, ppm) δ: 19.2 (CH₃, ═CCH₃), 51.5 (CH₃, OCH₃), 52.1 (CH₃, OCH₃), 52.2 (CH₃, OCH₃), 52.2 (CH₃, OCH₃), 125.0, 125.1 (CH, phen-CH³, phen-CH⁸), 126.9, 127.2 (CH, phen-CH⁵, phen-CH⁶), 127.3 (C, ═CCH₃), 129.6, 129.7 (C, phen-C¹³, phen-C¹⁴), 132.4 (C, C═C), 138.0, 138.2 (CH, phen-CH⁴, phen-CH⁷), 141.8 (C, C═C), 145.3, 146.9 (C, phen-C¹¹, phen-CH¹²), 149.6 (CH, phen-CH⁹), 154.1 (CH, phen-CH²), 161.3 (C, CO), 167.3 (C, CO), 167.6 (C, PdC═), 170.1 (C, CO), 173.5 (C, CO).

Characteristic IR peaks: ν_{(CO)} 1716, 1728 cm⁻¹, tablet in KBr

### Example 6: Preparation of the Compound (VI)

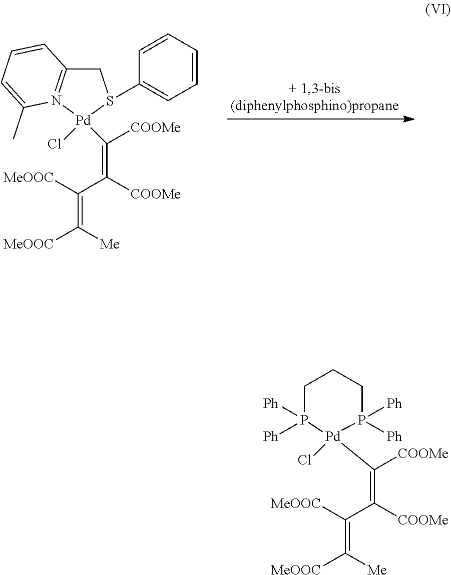

In a 100 mL tailed flask, 0.0820 g (0.1249 mmol) of [(Me-PyCH2SPh) Pd (ZC═CZ—CZ ═CZMe) Cl] and 0.0584 g (0.1374 mmol) of 1,3-bis (diphenylphosphino) propane (97% pure) dissolved in 12 mL of anhydrous dichloromethane. The solution, orange in color, is left under stirring for about 30 minutes, at the end of which it is reduced to a small volume on the rotary evaporator. The precipitation of the product is induced by the addition of n-hexane; the resulting orange solid is separated by filtration on gooch and dried in a vacuum pump. 0.0991 g (0.1161 mmol) of compound were obtained, equal to a yield of 93%.

The compound was characterized by NMR and IR spectroscopy.

Below is the listing and the attribution of the peaks in the ^{1H}-NMR, ¹³C {1H}-NMR and ³¹P {1H}-NMR spectra.

¹H-NMR (300 MHz, CDCl₃, T=298 K, ppm) δ: 1.18 (m, 3H, ═CCH₃), 2.17-3.00 (m, 6H, CH₂), 3.51 (s, 3H, OCH₃), 3.65 (s, 3H, OCH₃), 3.78 (s, 3H, OCH₃), 3.83 (s, 3H, OCH₃), 7.02-7.86 (m, 30H, Ph).

¹³C{¹H}-NMR (T=298K, CDCl₃, ppm) selected peaks, δ:16.4 (CH₃, ═CCH₃), 22.1 (CH₂, CH₂ central), 26.1 (dd, CH₂, J_{CP}=22.6, 6.4 Hz, PCH₂), 25.9 (dd, CH₂, J_{CP}=29.8, 7.2 Hz, PCH₂), 51.4 (CH₃, OCH₃), 51.6 (CH₃, OCH₃), 52.2 (CH₃, OCH₃), 53.1 (CH₃, OCH₃), 164.7 (d,C, J_{CP}=14.8, CO), 168.4 (C, CO), 170.2 (C, CO), 172.8 (C, CO), (the four signals C═C are not detectable).

³¹P{¹H}-NMR (T=298K, CDCl₃, ppm) δ: −5.0 (d, J_{CP}=45.5 Hz), 11.3 (d, J_{CP}=49.1 Hz)

Characteristic IR peaks: ν_{(CO)} 1713 cm⁻¹, tablet in KBr

### Example 7: Preparation of Compound (VII)

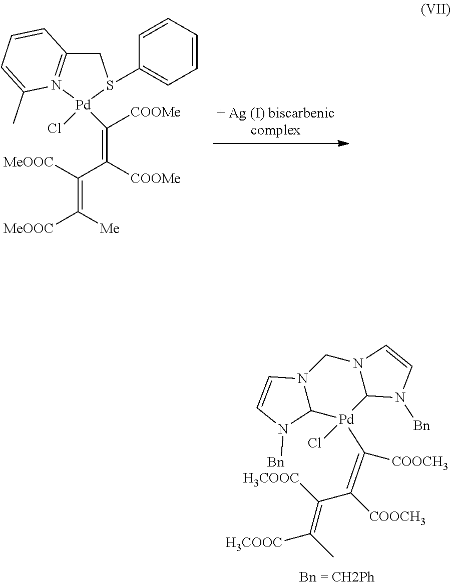

5 mL of anhydrous dichloromethane and 0.0154 g (0.0234 mmol) of [(Me-PyCH2SPh) Pd (ZC═CZ—CZ═CZMe) Cl] are placed in a 50 mL tailed flask, in an inert atmosphere (Ar). To this solution were added 0.0144 g (0.0234 mmol) of the Ag (1) biscarbenic complex (i.e. 1,1′-dibenzyl-3,3′-methylenediimidazole-2,2′-diylidene complex with Silver), dissolved separately in about 10 mL of anhydrous dichloromethane. The mixture was stirred for about 1 hour, during which the precipitation of AgCl is progressively observed. The latter was subsequently filtered on millipore. The orange solution obtained was reduced to a small volume in the rotary evaporator and the final product precipitated by adding diethyl ether. 0.0176 g of complex (brownish solid) were obtained, equal to a yield of 97%.

The compound was characterized by NMR and IR spectroscopy.

Below is the listing and the attribution of the peaks in the ¹H-NMR and ¹³C {1H}-NMR spectra.

¹H-NMR (300 MHz, CDCl₃, T=298 K, ppm) δ: 2.34 (s, 3H, ═CCH₃), 3.37 (s, 3H, OCH₃), 3.56 (s, 3H, OCH₃), 3.74 (s, 3H, OCH₃), 3.79 (s, 3H, OCH₃), 4.79-6.29 (AB system, J=14.8 Hz, 2H, CH₂Ph), 5.09-6.27 (AB system, J=15.0 Hz, 2H, CH₂Ph), 5.42-6.75 (AB system, J=13.2 Hz, 2H, NCH₂N), 6.51 (d, J=2.1 Hz, 1H, CH═CH^{Im}), 6.65 (d, J=1.9 Hz, 1H, CH═CH^{Im}), 6.90 (d, J=1.9 Hz, 1H, CH═CH^{Im}), 6.91 (d, J=2.1 Hz, 1H, CH═CH^{Im}), 7.21-7.35 (10H, Ar—H).

¹³C{¹H}-NMR (T=298K, CDCl₃, ppm) δ: 20.9 (CH₃, ═CCH₃), 51.3 (CH₃, OCH₃), 51.6 (CH₃, OCH₃), 52.2 (CH₃, OCH₃), 54.3 (CH₂, CH₂Ph), 54.9 (CH₂, CH₂Ph), 63.6 (CH₂, NCH₂N), 119.5 (CH, CH═CH^{Im}), 119.8 (CH, CH═CH^{Im}), 120.7 (CH, CH═CH^{Im}), 121.0 (CH, CH═CH^{Im}), 127.0-146.1 (Ar—C, C═C), 163.9 (C, CO), 165.3 (C, CO), 166.0 (C, carbene), 169.6 (C, PdC═), 172.6 (C, CO), 175.0 (C, carbene), 175.8 (C, CO).

Characteristic IR peaks: ν_{(CO)} 1716, 1728 cm⁻¹, tablet in KBr.

### Example 8: Preparation of Compound (VIII)

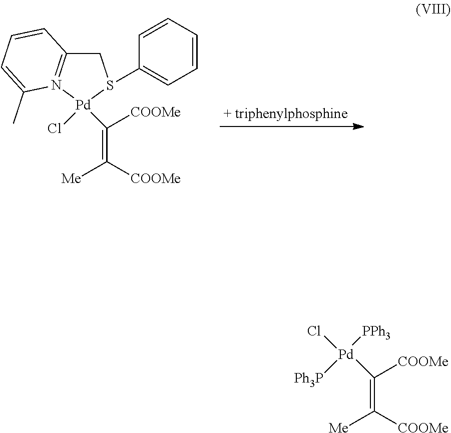

In a 100 mL tailed flask, in an inert atmosphere (Ar), 10 mL of anhydrous dichloromethane, 0.0820 g (0.159 mmol) of [(Me-PyCH2SPh) Pd (ZC═CZMe) Cl] and 0.0920 g are inserted (0.351 mmol) of triphenylphosphine. The mixture was reacted for 1 h at room temperature. Finally, the volume of the rotary evaporator is reduced and product precipitation is induced by adding n-hexane. By filtering on gooch, a solid yellow color is obtained. 0.112 g (0.136 mmol) of product were obtained, equal to a yield of 86%.

The compound was characterized by NMR and IR spectroscopy.

Below is the listing and the attribution of the peaks in the ¹H-NMR and ³¹P {1H}-NMR spectra.

¹H-NMR (300 MHz, CDCl₃, T=298 K, ppm) δ: 7.72 (m, 12H, Ar—H), 7.40 (m, 18H), 3.42 (s, 3H, COOCH₃α), 3.13 (s, 3H, COOCH₃μ), 1.22 (t, 3H, ═C—CH₃, J_{PH}=1.2 Hz).

³¹P{¹H}-NMR (CDCl₃, T=298 K, ppm) δ: 26.00.

Characteristic IR peaks: ν_{(CO)} 1709 cm⁻¹; tablet in KBr.

### Example 9: Preparation of Compound (IX)

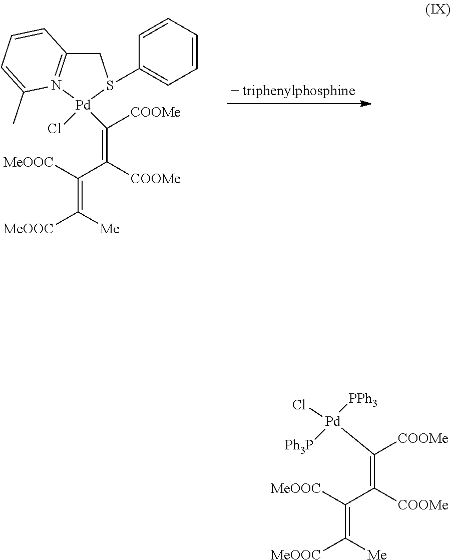

In a 100 mL tailed flask, in an inert atmosphere (Ar), 10 mL of anhydrous dichloromethane, 0.1000 g (0.152 mmol) of [(Me-PyCH2SPh) Pd (ZC═CZ—CZ═CZMe) Cl] and 0.0880 g (0.336 mmol) of triphenylphosphine. The mixture was reacted for 1 hour at room temperature. Finally, the solvent is removed from the rotary evaporator, the compound is dissolved in diethyl ether and the product precipitates by adding n-hexane. By filtering on gooch, a pink solid is obtained. 0.1370 g (0.142 mmol) of product were obtained, equal to a yield of 93%.

The compound was characterized by NMR and IR spectroscopy.

Below is the listing and the attribution of the peaks in the ¹H-NMR and ³¹P {1H}-NMR spectra.

¹H-NMR (300 MHz, CDCl₃, T=298 K, ppm) δ: 7.68 (m, 12H, Ar—H), 7.37 (m, 18H), 3.74 (s, 3H, COOCH₃), 3.56 (s, 3H, COOCH₃) 3.33 (s, 3H, COOCH₃), 2.94 (s, 3H, COOCH₃), 0.82 (t, 3H, ═C CH₃).

³¹P{¹H}-NMR (CDCl₃, T=298, ppm) δ: 20.5.

Characteristic IR peaks: ν_{(CO)} 1709 cm⁻¹; tablet in KBr.

### Example 10: Preparation of Compound (X)

This compound was prepared according to the method reported in the literature (Luciano Canovese, Fabiano Visentin, Gavino Chessa, Paolo Uguagliati, Claudio Santo, Alessandro Dolmella, Organometallics 2005, 24, 3297-3308, in particular on page 3304, diagram 6).

Example 11: evaluation of the therapeutic efficacy of the compounds of the invention.

Tests were carried out to determine the inhibitory concentration (IC50) of the compounds of the invention against tumor cell lines and on a control cell line; for comparison, the same tests were carried out with the cisplatin compound.

The cell lines tested were: A2780 and A2780cis, (ovarian cancer), MDA-MB231 (triple-negative breast cancer) and DLD1 (colon cancer) and MRC-5 (control; lung fibroblasts).

The stock solutions were obtained in dimethyl sulfoxide for compounds I-X and in H²⁰ for cisplatin.

The cells were kept, in accordance with the protocols indicated by the suppliers, at 37° C. in an atmosphere containing 5% carbon dioxide. About 500 cells were plated on 96 different wells and kept under such conditions for 96 hours.

Cell viability was determined, after treatment of the cells with a 1:1 solution of CellTiter-Glo® (registered trademark of Promega Corp., Madison, Wisconsin, USA) and DPBS, with the Tecan Infinite® M1000 instrument. IC₅₀ values were calculated from dose response curves. The means and standard deviations were obtained from tripled measurements.

Example 12: Comparison between known compound 1a and compound (VII) of the invention

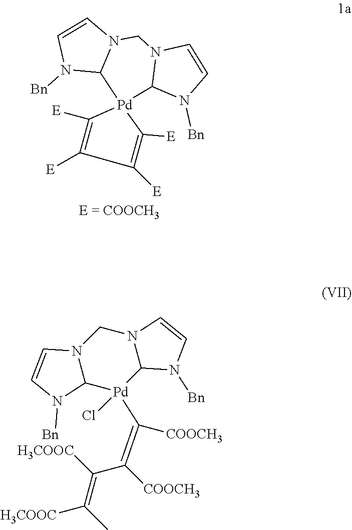

Compound 1a is disclosed by Thomas Scattolin, Isabella Caligiuri, Nayla Mouwad, Maguie El Boustani, Nicola Demitri, Flavio Rizzolio, Fabiano Visentin, in European Journal of Medicinal Chemistry, 179 (2019), 325-334.

Compound of formula (VII) of the present patent application contains a chelating biscarbene ligand, the Pd-butadienyl fragment and a chloride ligand. As reported in Table I, this compound exhibits good cytotoxicity towards ovarian cancer and triple-negative breast cancer lines. This cytotoxicity appears to be selective towards tumor cells as the compound is substantially inactive towards MRC-5 normal cells.

In the above mentioned previous publication, our group described a complex containing the same chelating biscarbene ligand, but with a palladacyclopentadienyl fragment (compound 1a).

Although to rigor, it is not entirely correct to make a comparison between the IC₅₀ absolute values of the two compounds, due to the different batches of tumor cells examined, it is however appropriate to underline that compound 1a significantly loses its antitumor activity passing from the A2780 line (cisplatin-sensitive ovarian cancer) to A2780cis (cisplatin-resistant ovarian cancer). More in detail, the ratio of IC₅₀ between the two lines is ca. 100, meaning a loss of activity comparable to that of platinum compounds, for which the efficacy in tumors that show resistance is very limited. Furthermore, an indirect evidence confirming the similar behaviour of 1a and cisplatin, is represented by the main molecular target which in both cases seems to be the nuclear DNA.

On the contrary, compound of formula (VII) of the present invention exhibits a more contained loss of activity between the two lines (ca. one order of magnitude), suggesting a different mechanism than compound 1a.

It should be noted that the realization of compounds with a mechanism of action different from the classical metallodrug-DNA interaction is of fundamental importance for the development of new generations of drugs for the treatment of cisplatin-resistant tumors.

A further confirmation of the great potential of compound VII and the other Pd-vinyl and Pd-butadienyl derivatives described here compared to the palladium analogues reported in the literature is the high cytotoxicity towards triple-negative breast cancer cells (see IC₅₀ values in Table I). This type of tumor is the most aggressive in panorama of breast cancer and is in most cases resistant to treatment with classical platinum-based antineoplastic agents.

Therefore, such a comparative example clearly shows the much better behaviour as anticancer agent of the compounds of the invention, as well as the inventiveness thereof.

1. A compound of formula (A) or formula (B) or formula (C): 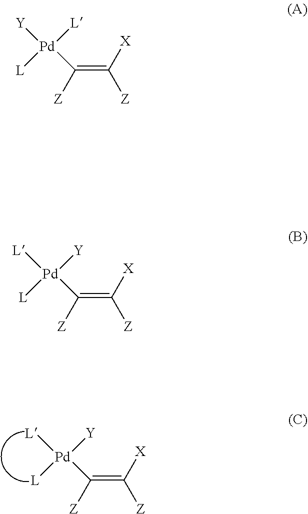
wherein:
Y is a halogen,
Z is COOR or R, where R is a linear or branched C1-C12 alkyl or aryl group,
X is a halogen, COOR or R, where R is a linear or branched C1-C12 alkyl or aryl group,
or X is C(Z)═C(Z)(X′) group, where Z is defined as above, X′ is in geometry trans with respect to Z and is a halogen, COOR or R, where R is a linear or branched C1-C12 alkyl or aryl group,
L and L′ are equal or different monodentate ligands, and
L-L′ is a bidentate ligand; for use as a medicament.

2. The compound according to claim 1, for use as an antitumor and/or in an antitumor treatment method.

3. The compound according to claim 1, for use in a method of treating tumors of the ovaries, cervix, colon or lungs.

4. The compound according to claim 1, wherein L and L′ are selected from the group consisting of: phosphines, phosphites, isonitriles, amines, thiols, alcohols, carbenes, alkyls, aryls, and carbonyls.

5. The compound according to claim 1, wherein L-L′ is selected from the group consisting of: diphosphines, diamines, glycols, biscarbenes, and bisisonitriles.

6. The compound according to claim 5, wherein:
Z is COOR, where R is a linear or branched C1-C12 alkyl group,
X is R, where R is a linear or branched C1-C12 alkyl group, or X is a group C(Z)═C(Z)(X′), where Z is COOR, where R is a linear or branched C1-C12 alkyl group and X′ in trans geometry with respect to Z is an a linear or branched C1-C12 alkyl group.

7. The compound according to claim 6, wherein:
Z is COOMe,
X is Me or X is a group C(Z)═C(Z)(X′), where Z is COOMe and X′ in trans geometry with respect to Z is Me.

8. The compound according to claim 1, wherein Y is chlorine.

9. The compound according to claim 1, wherein:
L and/or L′ are chosen from 1,3,5-triaza-7-phosphoadamantane and triphenylphosphine,
or,
L-L′ is 1,10-phenanthroline, 1,3-bis(diphenylphosphino)propane, 1,2-bis (diphenylphosphino)ethane, or 1,1′-dibenzyl-3,3′-methylenediimidazole-2,2′-diylidene.

10. The compound according to claim 1, selected from the group of the following compounds:

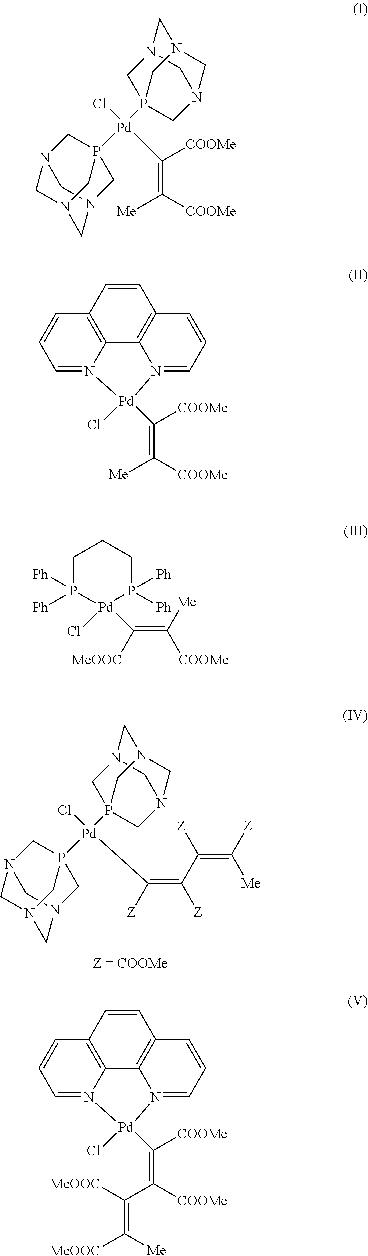

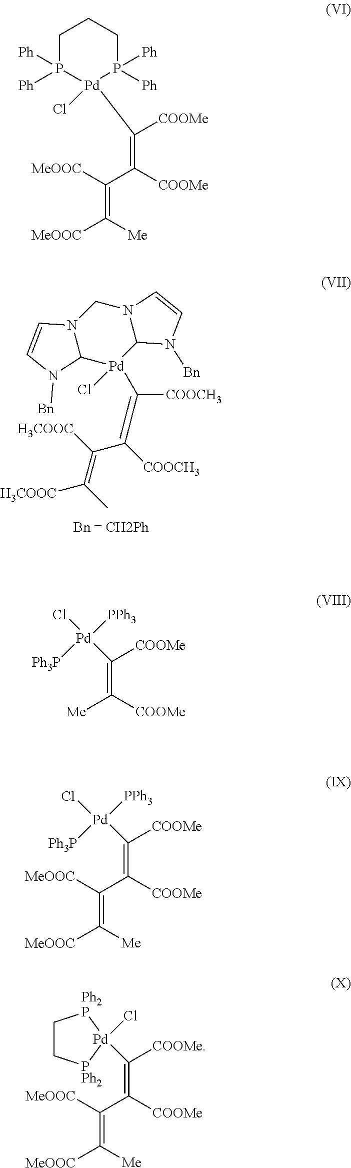

11. A pharmaceutical composition comprising a compound according to claim 1, and at least one pharmaceutically acceptable excipient.

12. A process for the preparation of a compound according to claim 1, comprising the reaction of a compound of formula (D): 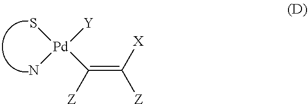
wherein:
Y is a halogen,
Z is COOR or R, where R is a linear or branched C1-C12 alkyl or aryl group,
X is a halogen, COOR or R, where R is a linear or branched C1-C12 alkyl or aryl group, or X is a C(Z)═C(Z)(X′) group, where Z is defined as above, X′ is in geometry trans with respect to Z and is a halogen, COOR or R, where R is a linear or branched C1-C12 alkyl or aryl group,
S—N is 2-methyl-6-((phenylthio)methyl)pyridine with at least one L or L′ monodentate ligand or with an L-L′ bidentate ligand.

13. The process according to claim 12, wherein in the compound of formula (D):
Z is COOR, where R is an alkyl group,
X is R, where R is a linear or branched C1-C12 alkyl group, or X is a group C(Z)═C(Z)(X′), where Z is COOR, where R is a linear or branched C1-C12 alkyl group and X′ in trans geometry with respect to Z is an a linear or branched C1-C12 alkyl group.

14. The process according to claim 13, wherein in the compound of formula (D):
Z is COOMe,
X is Me or X is a group C(Z)═C(Z)(X′), where Z is COOMe and X′ in trans geometry with respect to Z is Me.

15. The process according to claim 12, wherein in compound (D) Y is chlorine.

16. The process according to claim 12, wherein the compound (D) is 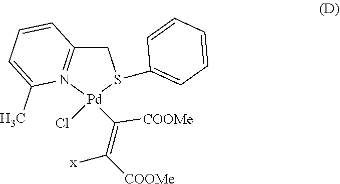
wherein X is Me or X is a group C(Z)═C(Z)(X′), where Z is COOMe and X′ in trans geometry with respect to Z is Me.

17. The process according to claim 12, wherein:
L and/or L′ are chosen from 1,3,5-triaza-7-phosphoadamantane and triphenylphosphine,
or,
L-L′ is 1,10-phenanthroline, 1,3-bis(diphenylphosphino)propane, 1,2-bis (diphenylphosphino)ethane, or 1,1′-dibenzyl-3,3′-methylenediimidazole-2,2′-diylidene.

18. The process according to claim 12, wherein the reaction is carried out in dichloromethane.

19. The process according to claim 12, wherein the reaction is carried out at a temperature ranging from 0° C. to 35° C. for a time comprising from 5 minutes to two hours.

20. A compound chosen from the group of the following compounds: 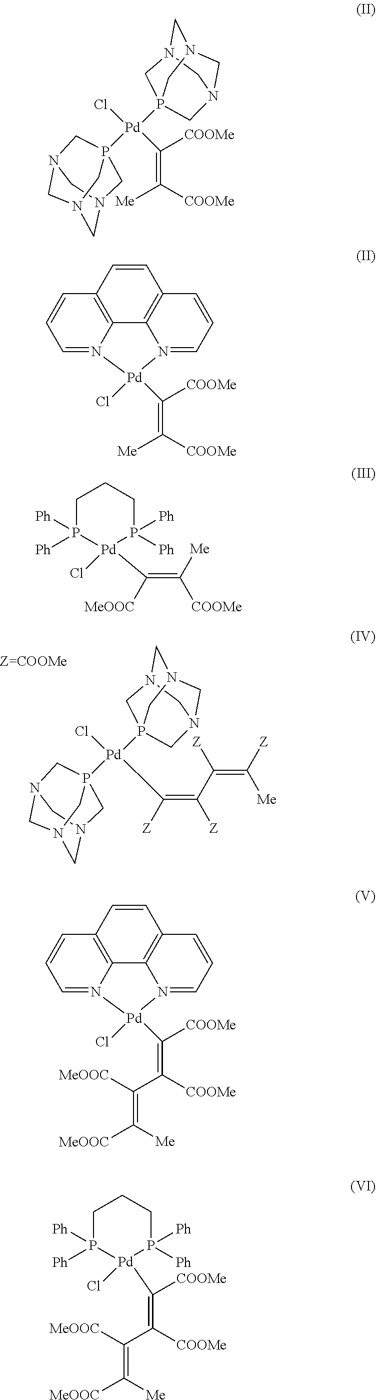 [chemical structure]
